(19) United States
(12) Patent Application Publication
(10) Pub. No.: US 2010/0239681 A1
(43) Pub. Date: Sep. 23, 2010

(54) Controlled Release Particulates Containing Water-Insoluble Drug

(75) Inventors: David Wong, Milpitas, CA (US); Xiufang Cheng, Fremont, CA (US); San-Laung Chow, San Jose, CA (US)

Correspondence Address: OCCHIUTI ROHLICEK & TSAO, LLP, 10 FAWCETT STREET, CAMBRIDGE, MA, 02138, US

(73) Assignee: Biokey, Inc., Fremont, CA (US)

(21) Appl. No.: 12/408,265

(22) Filed: Mar. 20, 2009

(51) Int. Cl. A61K 9/14; A61K 31/05; A61P 29/00

(52) U.S. Cl. 424/497; 514/733

(57) ABSTRACT

A pharmaceutical composition. The composition has a plurality of controlled release particulates. Each particulate includes a coating layer and a core partially coated with the coating layer, in which the core contains a water-insoluble drug 5-80 wt. % and a first polymer 0.2-80 wt. % and the coating layer contains the water-insoluble drug 0-50 wt. % and a second polymer 0.2-50 wt. %. Each of the first and second polymers, independently, is a water-insoluble polymer, an enteric polymer, or a combination thereof. A method of making the composition is also disclosed herein.

### BACKGROUND OF THE INVENTION

Controlled release formulations are widely used for slow, prolonged, or delayed release of medicaments after administration to a subject. The formulations are typically in the form of a coated pellet, a coated tablet, and a capsule. The release of the active medicaments is either effected through controlled permeability or selective erosion of the coating.

Various coating techniques (e.g., dipping, spraying, perforated pan coating, and fluid bed coating) have been applied to produce controlled release formulations. Spraying coating in a Wurster column is widely employed. In this technique, medicament particles are suspended in air while a coating solution/suspension is sprayed onto them. During the coating process, the solvent is constantly removed so that the coated particles are simultaneously dried to minimize agglomeration. A large amount of a solvent is often needed for preparing the coating solution/suspension and the solvent is subsequently removed during the coating process.

There is great interest in developing methods of preparing controlled release formulations.

### SUMMARY

This invention is based at least in part on a discovery that a pharmaceutical composition prepared by a novel method unexpectedly exhibits a rapid onset followed by a controlled release of the medicament in the composition.

In one aspect, this invention features a pharmaceutical composition having a plurality of controlled release particulates. Each of the particulates includes a coating layer and a core partially coated with the coating layer. The core contains a water-insoluble drug 5-80 wt. % and a first polymer 0.2-80 wt. % and the coating layer contains the water-insoluble drug 0-50 wt. % and a second polymer 0.2-50 wt. %, each of the first and second polymers, independently, being a water-insoluble polymer, an enteric polymer, or a combination thereof The composition is administered orally.

The weight percentages used herein are based on the total weight of each particulate. The term “water-insoluble drug” refers to a drug whose solubility in water is less than 0. 1 g/L at 25° C. One example of a water-insoluble drug is resveratrol (water solubility <0.03 g/L at 25° C.). The term “water-insoluble polymer” refers to a polymer whose solubility in water or an aqueous solution is less than 0.1 g/L at 25° C. and is substantially independent from the acidity of the aqueous solution. In other words, the water-insoluble polymers used in this invention are insoluble in the stomach or the intestine. The term “enteric polymer” refers to a polymer that is preferentially soluble in the less acidic environment of the intestine relative to the more acidic environment of the stomach.

Note that more than one drug can be included in the composition of the invention, provided that at least one drug is water-insoluble. For example, the composition includes both resveratrol and quercetin. In a preferred embodiment, the water-insoluble drug is predominantly found in the core of the particulate (e.g., 10-50 wt. %) and the coating layer is substantially free of the water-insoluble drug (e.g., 0-0.1 wt. %).

A water-insoluble drug refers to the drug compound itself, its salts, its solvates, and its prodrugs, if applicable. A salt, for example, can be formed between an anion and a positively charged group (e.g., amino) on a compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, mesylate, succinate, fumarate, tartrate, tosylate, salicylate, lactate, naphthalenesulfonate, and acetate. Likewise, a salt can also be formed between a cation and a negatively charged group (e.g., carboxylate) on a compound. Suitable cations include sodium ion, potassium ion, magnesium ion, calcium ion, and an ammonium cation such as tetramethylammonium ion. The drug compounds also include those salts containing quaternary nitrogen atoms. Examples of prodrugs include esters and other pharmaceutically acceptable derivatives, which, upon administration to a subject, are capable of providing active forms of the drugs described herein (see Goodman and Gilman's, The Pharmacological basis of Therapeutics, 8^{th} ed., McGraw-Hill, Int. Ed. 1992, “Biotransformation of Drugs”). In addition, drug compounds having asymmetric centers or a non-aromatic double bond, can occur as racemates, racemic mixtures, single enantiomers, individual diastereomers, diastereomeric mixtures, and cis- or trans-isomeric forms. For example, resveratrol refers to the compound itself, its isomer, and its pharmaceutically acceptable salts.

In the above-described controlled release particulates of the composition, the first and the second polymers can be the same or different. In one embodiment, each of the first and second polymers, independently, is a water-insoluble polymer such as polyacrylate (e.g., ethyl acrylate methyl methacrylate copolymer, polymethacrylate, polymethacrylic acid, or polyacrylic acid), cellulose ester (e.g., mono-, di-, or tri-cellulose acylates such as cellulose acrylate, cellulose acetate, cellulose acetate butyrate, cellulose diacetate, and cellulose triacetate), cellulose ether (e.g., ethyl cellulose), polyoxide (e.g., Carbowax), polyethylene, polypropylene, polyurethane, or a combination thereof (e.g., a mixture, a copolymer, and an alloy of polymers). Alternatively, each of the polymers, independently, can be an enteric polymer such as cellulose acetate phthalate, methyl acrylate-methacrylic acid copolymer, cellulose acetate succinate, hydroxyl propyl methyl cellulose phthalate, hydroxyl propyl methyl cellulose acetate succinate, polyvinyl acetate phthalate, methyl methacrylate-methacrylic acid copolymer, or a combination thereof (e.g., a mixture, a copolymer, and an alloy of polymers). In yet another embodiment, one of the polymers is a water-insoluble polymer and the other is an enteric polymer. Preferably, the core contains the first polymer 1-30 wt. % (e.g., 5-25 wt. % or 6-15 wt. %) or the coating layer contains the second polymer 0.5-30 wt. % (e.g., 5-25 wt. % or 6-15 wt. %).

The controlled release particulates may also have one or more of the following features. The coating layer constitutes 0.5-30 wt. % (e.g., 5-25 wt. % or 6-15 wt. %), with the balance being the core. It can further include an anti-sticking agent. Preferably, each particulate is no greater than 1500 microns (e.g., ≦1000 microns or ≦850 microns) in diameter. The term “diameter” refers to the distance between the two longest points on a particulate.

In another aspect, this invention features a method of preparing a controlled release pharmaceutical composition. The method includes providing a plurality of particles containing a water-insoluble drug (e.g., resveratrol) and a first polymer, coating the particles with a second polymer in a solvent to obtain wet coated particulates, drying the wet coated particulates to obtain dried coated particulates, and breaking the dried coated particulates to produce partially-coated particulates. The partially-coated particulates thus obtained each includes a coating layer and a core partially coated with the coating layer, the core containing the water-insoluble drug 5-80 wt. % and the first polymer 0.2-80 wt. %, and the coating layer containing the water-insoluble drug 0-50 wt. % and the second polymer 0.2-50 wt. %. Each of the first and second polymers, independently, is a water-insoluble polymer, an enteric polymer, or a combination thereof. It is preferred that each of the partially-coated particulates is no greater than 1500 microns (≦1000 microns) in diameter.

In the above-described method, the coating step can be performed in a high-shear mixer (e.g., a vertical mixer such as a planetary mixer, or a similar “bowl-type” mixer) and the breaking step can be performed by forcing the dried coated particulates to pass through a mesh screen either manually or by using a machine, e.g., a granulator.

The particles containing the water-insoluble drug and the first polymer can be obtained by three steps: mixing the water-insoluble drug, the first polymer, and a solvent (either the same as or different from the solvent for the second polymer) to form a wet mixture, passing the wet mixture through a first mesh screen to obtain wet granules (e.g., ≦2000 microns or ≦1500 microns in diameter), and then drying the wet granules to obtain the particles. When a high-shear mixer is used to perform the mixing step, it is preferred that the weight ratio of the solvent to the polymer used is 2:1 to 5:1. The thus obtained particles are preferably coated after sizing. Sizing can be achieved by forcing the dried granules to pass through a second mesh screen to obtain particles no greater than 1500 microns (e.g., ≦850 microns) in diameter.

Also with the scope of this invention is a controlled release pharmaceutical composition prepared according to the method described herein. Further contemplated is use of the above-described composition containing resveratrol via oral administration to treat inflammation, cancer, or neurodegenerative diseases.

An advantage of the pharmaceutical composition of this invention is that its rapid onset allows a quick alleviation of symptoms of the target disorder while the following controlled release phase allows a prolonged therapeutic effect. It is an unexpected finding that the composition having such an advantageous release profile was prepared by an inexpensive, reliable, and efficient method.

The details of one or more embodiments of the invention are set forth in the description below. Other features, objects, and advantages of the invention will be apparent from the description and from the claims.

### DETAILED DESCRIPTION OF THE INVENTION

The specific release profile, i.e., a rapid onset followed by a controlled release, exhibited by the pharmaceutical composition of this invention is determined by many factors, e.g., the configuration of the controlled release particulate, the nature of the drug, and the polymers used. As to the particulate configuration, it has a partially-coated core. More specifically, a certain amount of the drug in the core is exposed. Consequently, a certain amount of the drug, despite its water insolubility, is immediately released from the core when the particulate comes in contact with the body fluid. For example, the pharmaceutical composition releases 10-60% (e.g., 20-50%) of the drug in the first two hours upon administration, and then releases the remainder of the drug at a slower and more constant rate. Accordingly, the composition can release 20-85% (e.g., 30-80%) of the drug at the fourth hour, 50-100% (e.g., 60-95%) at the twelfth hour, and 70-100% (e.g., 75-100%) at the twenty-fourth hour.

The amount of the drug rapidly released depends on the size of the uncoated area of the core. Further, it can be adjusted upward by increasing the amount of the drug in the coating layer or including uncoated particulates in the composition.

The controlled release phase following the rapid onset phase is attributable to the nature of the drug and of the polymers contained in the core and the coating layer. As the drug is water insoluble and each of the first and second polymers independently is water insoluble or enteric, the body fluid can only slowly permeate or erode the polymers in the coating layer and the core to reach the drug embedded in the polymers so that the drug can be slowly released. The duration and release rate of the controlled release phase can be predetermined by using different polymers and drugs in varying amounts in the particulate, and controlling the size of the particulate. For instance, a higher amount of the polymers in the particulate leads to a slower release of the drug, and a smaller size of the particulate shortens the controlled release phase. Preferably, the controlled release phase lasts for about one day, i.e., 18-24 hours. To control the release rate, one can also include in the core a filler (e.g., 0.1-80 wt. % or 0.1-60 wt. %). Examples of a filler include but are not limited to lactose and ethyl cellulose.

The pharmaceutical composition can further include fully-coated particulates or uncoated particulates. Accordingly, the release profile of the composition can be adjusted by varying the amounts of fully-coated and uncoated particulates in the composition. More specifically, an increase in the amount of fully-coated particles results in a prolonged controlled release phase while an increase in the amount of uncoated particles brings about a more rapid onset.

In one embodiment, the water-insoluble drug used to practice this invention is resveratrol, a phytochemical produced by several plants. The water solubility of resveratrol is very poor, i.e., 0.03 g/L, at ambient temperature. It has been reported that resveratrol can be used to treat inflammation, cancer and other chronic diseases. See, e.g., U.S. Pat. Nos. 7,455,860, 7,153,877, and 7,037,945. It is also known for adversely altering xenobiotic metabolizing enzymes. The alteration depends on the dose, duration, and administration route. See, e.g., Canistro D. et al, Food Chem Toxicol, 2009, 47(2), page 454-61. A controlled release of resveratrol, which provides a more consistent drug plasma concentration, can reduce the just-mentioned adverse effect. Resveratrol can be used either in a pure form or in a plant extract.

To prepare the particulates of this invention, one can first mix a water-insoluble drug, a solvent, a first polymer, and optionally a filler at the desired ratio to form the cores of the particulates using conventional techniques, such as rotor-granulation, exclusion, fluid bed coating, perforated pan coating, and air-suspension. A high-shear mixer (which includes a mixing chamber, an impeller, and a chopper) can also be used. Once the cores are formed, they are then coated with a coating composition including another solvent, a second polymer, and optionally an anti-sticking agent. The two solvents used, each independently, can be an aqueous solvent or an organic solvent (e.g., acetone or isopropyl alcohol).

An example of how to prepare the particulates of this invention follows. A wet mixture containing resveratrol and a polyacrylate is first formed in an aqueous solvent using a high-shear mixer and is then forced through a 12-mesh screen (i.e., with 1500-micron openings) to form wet granules. After the wet granules are dried in an oven, they are forced to pass through a 20-mesh screen (i.e., with 850-micron openings) to obtain particles no greater than 850 microns in diameter. Next, the particles are placed in a high-shear mixer with a coating composition containing a polyacrylate. If preferred, an anti-sticking agent (e.g., magnesium stearate, glyceryl monostearate, or talc) and a plasticizer (e.g., acetyltributyl citrate, triacetin, acetyltriethyl citrate, dioctylphthalate, dibutylphthalate, triethyl citrate, tributylcitrate, polyethylene glycol, or propylene glycol) can be included in the coating composition in the amounts of 0.1-15 wt. % (e.g., 0.5-10 wt. %) of the whole particle and 0.01-30 wt. % (e.g., 0.1-15 wt. %) of the particle coating, respectively. Finally, the coated particles are dried in an oven at 40-60° C. for at least 3 hours and subsequently forced through an 18-mesh screen (i.e., with 1000-micron openings) to afford partially-coated particulates. In other words, when the coated particles are forced through the mesh screen, they are broken and, as a result, their coatings are partially removed. If desired, more than one coating layers can be applied to the core by repeating the relevant steps described above.

The pharmaceutical composition of this invention, when administered orally to a subject in need thereof, is preferably encapsulated in a capsule (e.g., a soft or hard-shell capsule). The capsule can be formed of a material that is well recognized by one skilled in the art, for example, porcine collagen material (e.g., porcine collagen or gelatin), bovine collagen material, gelatin, gum arabic, pectin, poly(ethylene-co-maleic anhydride), poly(vinvlmethylether-co-maleic anhydride), carrageenan, and agar-agar. Alternatively, the composition can also be mixed with conventional solid excipients and compressed to form tablets.

The release profile of the composition can be determined following a standard procedure known in the art. For example, the release rate of resveratrol is determined according to the United States Pharmacopeia (USP) 26 standard procedures employing a Type 2 Apparatus, at 50 rpm with 0.1 N HCl containing 1.5% w/w sodium lauryl sulfate (SLS) at 37° C. The release profile thus obtained resembles the in vivo release profile.

Without further elaboration, it is believed that the above description has adequately enabled the present invention. The following examples are, therefore, to be construed as merely illustrative, and not limitative of the remainder of the disclosure in any way whatsoever. All of the publications cited herein are hereby incorporated by reference in their entirety.

### EXAMPLE 1

A formulation was prepared via the following steps: (1) mixing 112.5 g of a resveratrol extract (purity 20%) with 4 g quercetin, and 81.5 g ethyl cellulose (EC) in a high shear granulator (Key International, KG5) to obtain a powder mixture; (2) adding 30 g Eudragit NE 30 D (an aqueous polyacrylate suspension, 30 wt. %, used as purchased) into the powder mixture during the mixing process to form wet granules; (3) forcing the wet granules to pass through a 12-mesh screen to remove granules greater than 1500 microns in diameter; (4) drying the wet granules at 55° C. until the loss-on-drying (LOD) was less than 3 wt. % of the dried granules; (5) forcing the dried granules to pass through a 20-mesh screen to remove granules greater than 850 microns in diameter and to afford the cores for the controlled release particulates; (6) coating the cores obtained above with a coating composition containing 45 g Eudragit NE30D (used as purchased), 1.5 g polyethylene glycol (PEG, purchased from Dow), and 1.5 g isopropyl alcohol (IPA) in the same high shear mixer to obtain wet coated particulates; (7) drying the coated particulates for at least 3 hours until LOD was less than 2% of the dried particulates; and (8) forcing the dried particulates to pass through a 18-mesh screen to afford the formulation having controlled release particulates. The formulation was then weighed, divided equally, and encapsulated in numerous capsules.

The weight of the formulation contained in each capsule was calculated. The per capsule amount of each of the ingredients constituting the core and coating layer of the controlled release particulates was calculated and listed in Table 1-1 below.

TABLE 1-1
Ingredients (mg/capsule)
Core

| | |
|---|---|
| Resveratrol, 20% | 225 |
| Quercetin | 8 |
| EC | 163 |
| Eudragit NE 30D* | 18 |

Coating

| | |
|---|---|
| Eudragit NE 30D* | 27 |
| PEG | 3 |

*the solid component of the suspension

In this experiment, big and gummy lumps were formed during the coating process, resulting in a low yield of the formulation.

Six capsules were randomly picked and tested for the release profile of the formulation in 0.1 N HCl containing 1.5% w/w SLS according to USP 26 with a Type 2 Apparatus at 50 rpm at 37° C. All of them exhibited an almost identical release profile, indicating the consistency of the method and uniformity of the formulation produced by the method. The average release results were listed in Table 1-2 below. As indicated in this table, release rate in the controlled release phase was very slow.

TABLE 1-2
Release results

| Time (hour) | Amount Released (%) |
|---|---|
| 0 | 0 |
| 2 | 34.51 ± 2.84 |
| 8 | 53.48 ± 1.63 |
| 12 | 55.38 ± 1.85 |
| 18 | 56.09 ± 0.74 |

### EXAMPLE 2

The formulation of Example 2 was prepared in a manner similar to that described in Example 1. For making the cores of the particulates contained in the formulation, 45 g resveratrol, 8 g quercetin, 150 g EC, and 45 g Eudragit NE 30 D were used. For coating the cores, 45 g Eudragit NE 30 D was used. The formulation thus obtained was then weighed, divided equally, and encapsulated in numerous capsules.

The weight of the formulation contained in each capsule was calculated. The per capsule amount of each of the ingredients constituting the core and coating layer of the controlled release particulates was calculated and listed in Table 2-1 below.

TABLE 2-1
Ingredients (mg/capsule)
Core

| | |
|---|---|
| Resveratrol | 45 |
| Quercetin | 8 |
| EC | 150 |
| Eudragit NE 30D* | 13.5 |

Coating

| | |
|---|---|
| Eudragit NE 30D* | 13.5 |

*the solid component of the suspension

In this experiment, there was no sticking problem during the core forming and coating processes. However, only loose granules could be obtained, resulting in a low yield of the formulation

The test for release profile was performed in a manner similar to that described in Example 1. The test results are shown in Table 2-2 below.

TABLE 2-2
Release results

| Time (hour) | Amount Released (%) |
|---|---|
| 0 | 0 |
| 2 | 22.72 ± 11.59 |
| 4 | 34.91 ± 12.69 |
| 8 | 50.18 ± 10.96 |
| 12 | 59.47 ± 7.59 |

### EXAMPLE 3

The formulation of Example 3 was prepared in a manner similar to that described in Example 1. For making the cores of the particulates contained in the formulation, 22.5 g resveratrol, 4 g quercetin, 40 g lactose, 10 g Eudragit NE 30 D, and 5 g IPA were used. For coating the cores, 17 g Eudragit NE 30 D and 5.7 g IPA were used. The formulation thus obtained was then weighed, divided equally, and encapsulated in numerous capsules.

The weight of the formulation contained in each capsule was calculated. The per capsule amount of each of the ingredients constituting the core and coating layer of the controlled release particulates was calculated and listed in Table 3-1 below.

TABLE 3-1
Ingredients (mg/capsule)
Core

| | |
|---|---|
| Resveratrol | 45 |
| Quercetin | 8 |
| Lactose | 80 |
| Eudragit NE 30D* | 6 |

Coating

| | |
|---|---|
| Eudragit NE 30D* | 10.2 |

*the solid component of the suspension

In this experiment, similar to what was observed in Example 1, sticky lumps were formed during the core forming and coating processes, resulting in a low yield of the formulation.

The test for release profile was performed in a manner similar to that described in Example 1. The test results are shown in Table 3-2 below.

TABLE 3-2
Release results

| Time (hour) | Amount Released (%) |
|---|---|
| 0 | 0 |
| 2 | 24.49 ± 7.40 |
| 4 | 37.50 ± 6.67 |
| 8 | 54.30 ± 5.27 |
| 12 | 65.58 ± 4.39 |

### EXAMPLE 4

The formulation of Example 4 was prepared in a manner similar to that described in Example 1. For making the cores of the particulates contained in the formulation, 45 g resveratrol, 8 g quercetin, 80 g lactose, 30 g Eudragit NE 30 D, and 15 g IPA were used. For coating the cores, 30 g Eudragit NE 30 D and 10 g IPA were used. The formulation thus obtained was then weighed, divided equally, and encapsulated in numerous capsules.

The weight of the formulation contained in each capsule was calculated. The per capsule amount of each of the ingredients constituting the core and coating layer of the controlled release particulates was calculated and listed in Table 4-1 below.

TABLE 4-1
Ingredients (mg/capsule)
Core

| | |
|---|---|
| Resveratrol | 45 |
| Quercetin | 8 |
| Lactose | 80 |
| Eudragit NE 30D* | 9 |

Coating

| | |
|---|---|
| Eudragit NE 30D* | 9 |

*the solid component of the suspension

The yield of the formulation is very low, as too many gummy lumps were produced in both the core forming and coating processes. However, a release profile was obtained in a manner similar to that described in Example 1. The test results are shown in Table 4-2 below.

TABLE 4-2
Release results

| Time (hour) | Amount released (%) |
|---|---|
| 0 | 0 |
| 2 | 21.86 ± 2.40 |
| 4 | 37.82 ± 1.60 |
| 8 | 59.39 ± 1.40 |
| 12 | 71.56 ± 0.95 |

### EXAMPLE 5

The formulation of Example 5 was prepared in a manner similar to that described in Example 1. For making the cores of the particulates contained in the formulation, 225 g resveratrol, 40 g quercetin, 400 g lactose, and 150 g Eudragit NE 30 D were used. For coating the cores, 162 g Eudragit NE 30 D was used. The formulation thus obtained was then weighed, divided equally, and encapsulated in numerous capsules.

The weight of the formulation contained in each capsule was calculated. The per capsule amount of each of the ingredients constituting the core and coating layer of the controlled release particulates was calculated and listed in Table 5-1 below.

TABLE 5-1
Ingredients (mg/capsule)
Core

| | |
|---|---|
| Resveratrol | 45 |
| Quercetin | 8 |
| Lactose | 80 |
| Eudragit NE 30D* | 9 |

Coating

| | |
|---|---|
| Eudragit NE 30D* | 9.7 |

*the solid component of the suspension

Unexpectedly, this experiment did not produce sticky lumps during the core forming and coating processes. Also unexpectedly, a satisfactory release profile (i.e., a rapid onset followed by a controlled release of resveratrol) was obtained as shown in Table 5-2.

TABLE 5-2
Release results

| Time (hours) | Amount Released (%) |
|---|---|
| 0 | 0 |
| 2 | 37.63 ± 8.29 |
| 4 | 57.16 ± 8.80 |
| 8 | 76.83 ± 6.27 |
| 12 | 84.73 ± 3.98 |
| 18 | 90.10 ± 2.04 |
| 24 | 92.27 ± 1.84 |

### EXAMPLE 6

The formulation of Example 6 was prepared in a manner similar to that described in Example 1. For making the cores of the particulates contained in the formulation, 45 g resveratrol, 8 g quercetin, 80 g lactose, 46.26 g Eudragit L30D-55 (a copolymer dispersion of methacrylic acid and ethyl acrylate, 30 wt. %, used as purchased), and 1.54 g triethyl citrate (TEC) were used. For coating the cores, 55.3 g Eudragit L30D-55 and 1.78 g TEC were used. The formulation thus obtained was then weighed, divided equally, and encapsulated in numerous capsules.

The weight of the formulation contained in each capsule was calculated. The per capsule amount of each of the ingredients constituting the core and coating layer of the controlled release particulates was calculated and listed in Table 6-1 below.

TABLE 6-1
Ingredients (mg/capsule)
Core

| | |
|---|---|
| Resveratrol | 45 |
| Quercetin | 8 |
| Lactose | 80 |
| Eudragit L30D-55 | 13.9 |
| TEC | 1.54 |

Coating

| | |
|---|---|
| Eudragit L30D-55 | 16.6 |
| TEC | 1.78 |

Eudragit L used in this example is an enteric polymer; which is not soluble in the acidic medium of the stomach (pH 1.2) while soluble at pH 6 or higher.

Unexpectedly, this experiment did not produce sticky lumps during the core forming and coating processes. Also unexpectedly, a satisfactory release profile (i.e., a rapid onset followed by a controlled release of resveratrol) was obtained as shown in Table 6-2 below.

TABLE 6-2
Release results

| Time (hours) | Amount Released (%) |
|---|---|
| 0 | 0 |
| 2 | 45.34 ± 17.47 |
| 4 | 56.12 ± 10.68 |
| 8 | 60.11 ± 15.02 |

### EXAMPLE 7

The formulation of Example 7 was prepared in a manner similar to that described in Example 1. For making the cores of the particulates contained in the formulation, 45 g resveratrol, 8 g quercetin, 80 g lactose, and 30.4 g Surelease (an aqueous ethyl cellulose dispersion, 25 wt. %, used as purchased) were used. For coating the cores, 30.7 g Surelease was used. The formulation thus obtained was then weighed, divided equally, and encapsulated in numerous capsules.

The weight of the formulation contained in each capsule was calculated. The per capsule amount of each of the ingredients constituting the core and coating layer of the controlled release particulates was calculated and listed in Table 7-1 below.

TABLE 7-1
Ingredients (mg/capsule)
Core

| | |
|---|---|
| Resveratrol | 45 |
| Quercetin | 8 |
| Lactose | 80 |
| Surelease | 7.6 |

Coating

| | |
|---|---|
| Surelease | 7.67 |

Unexpectedly, this experiment did not produce sticky lumps during the core forming and coating processes. Also unexpectedly, a satisfactory release profile (i.e., a rapid onset followed by a controlled release of resveratrol) was obtained as shown in Table 7-2 below.

TABLE 7-2
Release results

| Time (hours) | Amount Released (%) |
|---|---|
| 0 | 0 |
| 2 | 43.90 ± 9.97 |
| 4 | 63.15 ± 6.55 |
| 8 | 78.17 ± 3.04 |
| 12 | 83.54 ± 2.86 |
| 18 | 85.11 ± 1.24 |

### Other Embodiments

All of the features disclosed in this specification may be combined in any combination. An alternative feature serving the same, equivalent, or similar purpose may replace each feature disclosed in this specification. For example, the core or the coating layer can further contain a water-soluble polymer such as hydroxypropyl methylcellulose, hydroxypropyl cellulose, polyvinylpyrrolidone to vary the release rate. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A pharmaceutical composition comprising a plurality of controlled release particulates, each particulate, including a coating layer and a core partially coated with the coating layer, wherein the core contains a water-insoluble drug 5-80 wt. % and a first polymer 0.2-80 wt. % and the coating layer contains the water-insoluble drug 0-50 wt. % and a second polymer 0.2-50 wt. %, each of the first and second polymers, independently, being a water-insoluble polymer, an enteric polymer, or a combination thereof.

2. The composition of claim 1, wherein the water-insoluble drug is resveratrol.

3. The composition of claim 2, wherein the coating layer contains resveratrol 0-0.1 wt. % and the second polymer 0.5-30 wt. %.

4. The composition of claim 3, wherein the coating layer constitutes 0.5-30 wt. % of the particulate.

5. The composition of claim 4, wherein the coating layer constitutes 5-25 wt. % of the particulate.

6. The composition o f claim 2, wherein the core further contains a filler 0.1-80 wt. %.

7. The composition of claim 2, wherein the core further contains a filler 0.1-60 wt. %.

8. The composition of claim 2, wherein each of the first and second polymers is a water-insoluble polymer.

9. The composition of claim 8, wherein the water-insoluble polymer is polyacrylate, cellulose ester, cellulose ether, polyoxide, polyethylene, polypropylene, polyurethane, or a combination thereof.

10. The composition of claim 9, wherein the water-insoluble polymer is ethyl acrylate methyl methacrylate copolymer.

11. The composition of claim 2, wherein the enteric polymer is cellulose acetate phthalate, methyl acrylate-methacrylic acid copolymer, cellulose acetate succinate, hydroxyl propyl methyl cellulose phthalate, hydroxyl propyl methyl cellulose acetate succinate, polyvinyl acetate phthalate, methyl methacrylate-methacrylic acid copolymer, or a combination thereof.

12. The composition of claim 1, wherein the coating layer contains the water-insoluble drug 0-0.1 wt. % and the second polymer 0.5-30 wt. %.

13. The composition of claim 12, wherein the coating layer constitutes 0.5-30 wt. % of the particulate.

14. The composition o f claim 1, wherein the core further contains a filler 0.1-80 wt. %.

15. The composition of claim 1, wherein the water-insoluble polymer is polyacrylate, cellulose ester, cellulose ether, polyoxide, polyethylene, polypropylene, polyurethane, or a combination thereof.

16. The composition of claim 15, wherein the water-insoluble polymer is ethyl acrylate methyl methacrylate copolymer.

17. The composition of claim 1, wherein the enteric polymer is cellulose acetate phthalate, methyl acrylate-methacrylic acid copolymer, cellulose acetate succinate, hydroxyl propyl methyl cellulose phthalate, hydroxyl propyl methyl cellulose acetate succinate, polyvinyl acetate phthalate, methyl methacrylate-methacrylic acid copolymer, or a combination thereof.

18. The composition of claim 2, wherein each of the plurality of controlled release particulates is not greater than 1500 microns in diameter.

19. The composition of claim 18, wherein each of the plurality of controlled release particulates is not greater than 1000 microns in diameter.

20. A method of preparing a controlled release pharmaceutical composition, comprising:
providing a plurality of particles containing a water-insoluble drug and a first polymer,
coating the particles with a second polymer in a solvent to obtain wet coated particulates,
drying the wet coated particulates to obtain dried coated particulates, and
breaking the dried coated particulates to produce partially-coated particulates, whereby the partially-coated particulates each includes a coating layer and a core partially coated with the coating layer, in which the core contains the water-insoluble drug 5-80 wt. % and the first polymer 0.2-80 wt. %, and the coating layer contains the water-insoluble drug 0-50 wt. % and the second polymer 0.2-50 wt. %, each of the first and second polymers, independently, being a water-insoluble polymer, an enteric polymer, or a combination thereof.

21. The method of claim 20, wherein the water-insoluble drug is resveratrol.

22. The method of claim 20, wherein the coating step is performed in a high-shear mixer.

23. The method of claim 22, wherein the weight ratio of the solvent to the first polymer is 2:1 to 5:1.

24. The method of claim 20, wherein the particles are obtained by
mixing the water-insoluble drug, the first polymer, and a solvent to form a wet mixture,
passing the wet mixture through a first mesh screen to obtain wet granules, and
drying the wet granules to obtain the particles.

25. The method of claim 24, wherein the particles thus obtained are further sized.

26. The method of claim 25, wherein the particles are sized by forcing the dried granules to pass through a second mesh screen.

27. The method of claim 24, wherein each wet granule is not greater than 2000 microns in diameter and each particle is not greater than 1500 microns in diameter.

28. The method of claim 24, wherein each wet granule is not greater than 1500 microns in diameter and each particle is not greater than 850 microns in diameter.

29. The method of claim 24, wherein the mixing step is performed in a high-shear mixer.

30. The method of claim 29, wherein the weight ratio of the solvent to the first polymer is 2:1 to 5:1.

31. The method of claim 20, wherein each partially-coated particulate is not greater than 1500 microns in diameter.

32. The method of claim 20, wherein each partially-coated particulate is not greater than 1000 microns in diameter.

33. The method of claim 20, wherein the breaking step is performed by forcing the dried coated particulates to pass through a mesh screen.

34. A controlled release pharmaceutical composition prepared according to the method of claim 20.
